(19) United States
(12) Patent Application Publication
(10) Pub. No.: US 2018/0103991 A1
(43) Pub. Date: Apr. 19, 2018

(54) DEVICE AND METHOD FOR TISSUE TREATMENT BY COMBINATION OF ENERGY AND PLASMA

(71) Applicant: BTL HOLDINGS LIMITED, Nicosia (CY)

(72) Inventors: Stanislav Linhart, Hradistko (CZ); Tomas Schwarz, Praha (CZ)

(21) Appl. No.: 15/786,303

(22) Filed: Oct. 17, 2017

Related U.S. Application Data
(60) Provisional application No. 62/409,665, filed on Oct. 18, 2016.

(51) Int. Cl. A61B 18/04; A61B 18/14

(52) U.S. Cl. CPC A61B 18/042; A61B 2018/00577; A61B 18/1477

(57) ABSTRACT

In methods and devices for treatment of tissue of a patient, one or more applicators is positioned adjacent to the tissue to be treated. A cold plasma and radiofrequency energy are applied to the tissue. A negative pressure may be used to draw the tissue towards and/or into contact with the energy delivery elements.

### PRIORITY CLAIM

The application claims priority from Provisional Application No. 62/409,665 filed on Oct. 18, 2016 which is incorporated by reference.

### FIELD OF INVENTION

The present invention relates to a device and method for treatment of tissue by using a plasma source in combination with another energy source. Treating tissue includes tissue healing, rejuvenation and adipose tissue reduction using a plasma and other energy source.

### BACKGROUND

Tissue is composed of a few layers. While the skin on the surface of the human or animal body includes epidermis, dermis, hypodermis and muscles located beneath the hypodermis, body cavities include layers which may be more or less differentiated. For example, tissue layers in the vaginal cavity include the epithelium, lamina propria (having similar composition to dermis), muscles and adventitia. The outer and also the thinnest layer (e.g. epithelium) of tissue is the epidermis. The second layer (e.g. dermis) includes connective tissue and reticular fibers. The third layer of the skin, the hypodermis, is the lowest layer of the skin and contains hair follicle roots, lymphatic vessels, collagen tissue, nerves and also fat forming a subcutaneous white adipose tissue. The adipose cells create lobules which are bounded by connective tissue and/or fibrous septa. Another type of adipose tissue is a visceral adipose tissue between organs beneath the muscles.

Currently used methods and devices enable treatment of a wide variety of tissue problems by using different types of energy, e.g. electromagnetic energy. However, these methods and devices may cause wounds, lesions and/or micro-injuries on and/or under the surface of the treated tissue. The wound healing may also be lengthy process and may be complicated by attack of pathogens and related immunological responses leading to inflammation. Another problem is lack of sufficient contact of the tissue with the applicator of such device, where the non-sufficient contact may lead to burning of tissue and/or nonhomogeneous treatment.

There is an increasing demand for aesthetics procedures to reduce or reverse effects caused e.g. by aging, sun exposure, injuries, dermatological diseases, or excess of adipose tissue. Skin aging may include different characteristics of connective tissue, which may lead to variety of tissue problems e.g. wrinkles, sagging, prolapse, laxity and/or stretch marks. Other tissue problems may include e.g. freckles, acne, rosacea, spots, pigment and skin lesions, cellulite, tissue enlargement, or tissue inflammation.

Plasma is an electrically neutral medium of unbound particles e.g. free electrons, ions, neutral atoms, molecules and radicals. Plasma may be ionized gas, but it should be noted, that plasma has different properties of that gas. Therefore, it may be described as fourth fundamental state of matter. Plasma may provide faster wound healing by its effects, e.g. bactericidal effect, activation or inhibition of receptors on the cell surface, stimulation of migration and proliferation of wound related tissue.

In the light of the above, there is a need for improved methods and devices using plasma for skin and tissue treatment and for wound healing.

### GLOSSARY

The term “tissue problem” means a scar, wrinkles, sagging, acne, rosacea, pigmented lesion, cellulite, skin lesion, stretch marks, hemangioma, hyperhidrosis, melasma, onychomycosis, excess of hair, spider vein, large skin pores, recalcitrant melasma, solar elastosis, mucinosis, amyloidosis, Hori's nevus, rosacea, excess of adipose tissue, open wound, closed wound, eczema, prolapse, laxity, enlargement, atrophy, erythrasma, impetigo contagiosa, tattoo, folliculitis, gram-negative foot infection, ecthymata and/or fungal infection. A tissue problem may be located in at least one layer of the tissue.

The term “energy” means any type of energy or field e.g. electromagnetic energy, electric energy, mechanical energy, thermal energy and/or magnetic field. The term “energy” also means the combination of at least two types of energy. Energy may be coherent and/or non-coherent

The term “direct contact” means any contact of the applicator of the device with the tissue or skin surface.

The term “indirect contact” means any contact of the applicator with the tissue through spacing object.

The term “no contact” means the applicator is spaced apart from the tissue by a gap.

The term “treated tissue” means the section of the skin surface and/or volume of the tissue influenced by the treatment.

The term “energy delivery element” means an electrode (e.g. capacitive, resistive and/or inductive), transducer, antenna, light source, photomixing source, resonant-tunneling diode, radionuclide and/or magnetic coil. The term “energy delivery element” also includes an energy providing element with additional parts; for example, an ultrasound transducer together with backing material, coupling liquid and acoustic window. The term “energy delivery element” also means an array of energy delivery elements.

The term “plasma source” means a source providing plasma in continuous and/or pulse mode. It may be a power source (e.g. radiofrequency, direct current, alternating current and/or microwave), where plasma may be generated by ionization of gas. It may be also DC or AC corona, steamer corona, cascaded arc plasma source, inductive coil and/or piezoelectric transformer. Plasma source also includes apparatus operating on the basis of a laser induced discharge, plasma jet, arc discharge, silent discharge and/or corona discharge, dielectric barrier discharge, capacitive discharge and generation by ionizing radiation which includes alpha, beta and gamma ray, ultraviolet light, X-rays and high energy electron beam.

### SUMMARY OF INVENTION

A combination of energy and plasma which may comprise cold plasma is used to treat tissue problems. Treatment may include only application of the plasma.

Combination of energy and plasma may be used for an aesthetic treatments focusing on tissue problems. More detailed methods of their treatment is disclosed below. The treatment methods may use at least one energy source and/or plasma source. Methods using electromagnetic energy may cause thermal damage within tissue which may induce contraction and/or at least partial destruction of tissue structure and/or adipose tissue reduction. As a result, treated or surrounding tissue may synthetize and/or repair of connective tissue. Delivering energy to the adipose tissue may induce apoptosis and/or necrosis of adipose cells, or fatty acid consumption, which may lead to reduction in the number and/or volume of adipose cells. After treatment the tissue may have an improved appearance. Furthermore mechanical waves which may be focused or unfocused may provide a similar effect on treated tissue and may have the same results as electromagnetic energy.

Thermal damage generated by energy transfer may include necrosis, apoptosis, ablation, coagulation, tightening and/or rejuvenation of the tissue. Other effects of the energy may be destruction, heating, dispersion, fragmenting and/or bleaching of the colored ink.

The apparatus and method provides improved healing of tissue, which is induced by plasma supplemented with another substance, e.g. nitric oxide, ozone, oxygen and its radicals.

A combination of radiofrequency (RF) energy with plasma supplemented with nitric oxide may provide effective tissue treatment with stimulation of regenerative process. Combination may provide improved healing process without risk of inflammation.

A combination of plasma and radiofrequency waves may be used, with the radiofrequency waves delivered to the tissue by needles. In that case, plasma may provide a healing and/or bactericide effect.

Negative and/or positive pressure may be applied to the tissue during treatment, where the pressure may ensure sufficient contact of the device with tissue, particularly in sites with thin tissue (e.g. face)

### BRIEF DESCRIPTION OF THE DRAWINGS

FIG. 1A is a diagram of an exemplary plasma source.

FIG. 1B is an example of exemplary plasma source.

FIG. 1C is another example of an exemplary plasma source.

FIG. 1D is another example of an exemplary plasma source.

FIG. 1E is another example of an exemplary plasma source.

FIG. 1F is another example of an exemplary plasma source.

FIG. 2A is an exemplary diagram of a treatment device

FIG. 2B is another example of a surface of the tip facing the tissue.

FIG. 2C is another example of a surface of the tip facing the tissue.

FIG. 2D is another example of a surface of the tip facing the tissue.

FIG. 2E is another example of a surface of the tip facing the tissue.

FIG. 2F is another example of a surface of the tip facing the tissue.

FIG. 3A is a diagram of a treatment device including needle electrodes.

FIG. 3B is a diagram of a exchangeable tip including retracted needle electrodes.

FIG. 3C another diagram of a exchangeable tip including extended needle electrodes.

FIG. 3D is a diagram of a treatment device including needle electrodes showing an example of thermal damage.

FIG. 3E is a diagram of a treatment device including needle electrodes showing another example of thermal damage.

FIG. 3F is a diagram of a treatment device including needle electrodes showing another example of thermal damage.

FIG. 3G is a diagram of a treatment device including needle electrodes showing another example of thermal damage.

FIG. 3H is a diagram of a treatment device including needle electrodes showing another example of thermal damage.

FIG. 4A is a diagram of a treatment device using negative pressure.

FIG. 4B is another diagram of a treatment device using negative pressure.

FIG. 4C is another diagram of a treatment device using negative pressure.

FIG. 5A is a diagram of a exchangeable tip including protruding elements.

FIG. 5B is another diagram of a treatment device using negative pressure.

FIG. 5C is another diagram of a treatment device using negative pressure.

FIG. 6A is an example of an exemplary thermal damage.

FIG. 6B is another example of an exemplary thermal damage.

FIG. 6C is another example of an exemplary thermal damage

### DETAILED DESCRIPTION

The method may provide treatment including application of the plasma and at least one form of energy to the tissue and/or adjacent to the tissue. Energy may be applied in pulses and/or in a continuous manner.

The electromagnetic energy may be ionizing energy (e.g. gamma radiation and/or X-rays), light (e.g. ultraviolet, visible and/or infrared light), terahertz energy, microwave energy and/or radiofrequency energy. Electromagnetic energy may include coherent and/or non-coherent energy.

The mechanical energy may be ultrasound energy, shock wave energy and/or vibrational energy. Mechanical energy may be targeted into the tissue in the form of focused, defocused and/or planar waves.

Shock wave energy may be generated by electrohydraulic, piezoelectric, electromagnetic and/or ballistic principles.

Optionally, applied energy may be magnetic field. A device and method for generation of a magnetic field as described in international patent application PCT/IB2016/053930 and U.S. patent application No. 62/357,679 incorporated herein by reference, may be used.

Effects of the applied energy may be enhanced by the application of a substance, e.g. a chromophore.

The application of plasma may be parallel and/or sequential to the application of the energy. Furthermore, application of plasma or energy may overlap with another application of plasma or energy.

Generally, plasma may include a partially ionized gas comprising free charge carriers e.g. free electrons, ions, neutral atoms, molecules and/or radicals. Plasma may be generated by applying sufficient energy to a volume of separate particles e.g. electrons from atoms and molecules. Plasma may be generated according to the following method. Electric field accelerates free electrons sufficiently to cause collisions with the gas molecules. The result is dissociation of electrons from the gas molecules to create gaseous ions, or the excitation of vibrational states. This process creates plasma. The energy of plasma declines by recombination of electrons and ions to form neutrally charged atoms or molecules. The energy released results in emission of electromagnetic radiation, wherein wavelength of the electromagnetic radiation depends on the gas used. The temperature of the plasma depends on the gas, and the amount of power, pressure and/or type of electric field used.

Types of plasma may be differentiated by temperature. High-thermal plasma may include electrons having the same temperature as temperature of ions and gas, therefore the temperature of the plasma is high. On the contrary, non-thermal plasma may include electrons having temperatures different from the temperature of the ions and gas. Special cases of non-thermal plasma may be cold plasma, where the ionized gas may have room temperature. Cold plasma is also characterized by high excitation selectivity and non-equilibrium chemical reactions. Another type of plasma is ultracold plasma generated by the source containing e.g. a laser.

Plasma may be generated by monopolar, bipolar, unipolar and/or multipolar energy delivery element. When the energy delivery element provides radiofrequency to generate plasma, the frequency of the radiofrequency may be in the range of 5 Hz to 1 GHz, more preferably in the range of 15 Hz to 500 MHz, most preferably in the range of 25 Hz to 15 MHz. In another configuration frequency of the radiofrequency may be in the range of 500 kHz to 6 GHz, more preferably 1 MHz to 4 GHz, most preferably in the range of 2 MHz to 3 GHz. Plasma may be generated by an energy delivery element also providing radiofrequency energy to the tissue where the application of the radiofrequency energy and plasma may be simultaneous or sequential to each other. When the plasma is produced by a voltage between two electrodes, the voltage may be in the range of 1 V to 30 kV, more preferably in the range of 1 kV to 30 kV, most preferably in the range of 1 kV to 20 kV. Voltage between electrodes may be about 220 V. Plasma may be generated in a specified duration referred as plasma pulse duration i.e. the time interval when the energy delivery element is generating plasma. Plasma pulse duration may be in the range of 0.1 nanoseconds to 30 seconds, more preferably in the range of 0.1 nanosecond to 5 s, even more preferably in the range of 0.5 nanosecond to 3 s, most preferably in the range of 1 nanosecond to 1 second. Temperature of non-thermal plasma when it reaches the tissue may be in the range of −50° C. to 150° C., more preferably in the range of −25° C. to 100° C., even more preferably in the range of −15° C. to 90° C., most preferably in the range of 0° C. to 85° C. Temperature of cold plasma when it reacts with the tissue may be in the range of 5° C. to 75° C., more preferably in the range of 20° C. to 55° C., even more preferably in the range of 30° C. to 50° C., most preferably in the range of 32° C. to 46° C. The distance of energy delivery element generating plasma from the surface of the tissue may be in the range of 0.1 mm to 15 cm, more preferably in the range of 0.5 mm to 12 cm, even more preferably in the range of 1 mm to 10 cm, most preferably in the range of 1 mm to 5 cm.

Plasma may be generated from a working medium, which may be gas, liquid (e.g. water) and/or chemical substance.

Plasma may be generated using gas. The gas used for generation of plasma may be referred to as source gas. The gas used for generation of plasma may be single type of gas e.g. argon, helium, nitrogen, oxygen. The gas may be also mixture of single types of gas e.g. air, mixture of argon and helium, argon and hydrogen, argon and oxygen, oxygen and hydrogen, nitrogen and oxygen, argon and oxygen or argon and oxygen together with nitric oxide. Oxygen may be used in order to produce plasma containing ozone and other reactive allotropes of oxygen e.g. tetraoxygen etc. The generated plasma may also interact with pure oxygen to create ozone.

Plasma may be supplemented with at least one substance before, during and/or after its generation. Supplied substances may include e.g. molecules, ions and/or radicals e.g. hydroxyl radical, peroxide radical, nitric oxide, ozone, carbon oxide, oxygen, ammonia. The substance may include a radical precursor and/or a radical scavenger. Nitric oxide and its radicals may be used for wound healing improvement. Ozone may be used for its bactericide effect and/or killing cancer cells. At least one radical (e.g. peroxynitrite superoxide, peroxide radical) may be supplied for enhancing bactericide effect. Supplied substances and/or plasma may also activate and/or inhibit at least one human protein e.g. gas sensor proteins may be activated.

Plasma may be used as a carrier for delivery of another gas e.g. nitric oxide or nitrous oxide. Alternatively, the source gas may be used as a carrier for delivery of another gas. Another gas delivered by plasma and/or source gas may be referenced as secondary gas. Source gas may be the same as secondary gas, but in that case the secondary gas includes other substances described below. Plasma may be used as a coolant. Furthermore, the source gas and/or secondary gas may be used as a coolant and/or anesthetic. The secondary gas may be a coolant (e.g. haloalkanes like R134a, R410A, carbon dioxide, hydrogen, nitrogen). Alternatively, the secondary gas may be anesthetic gas or it may include one or more anesthetic (e.g. lidocaine, procaine, prilocaine, benzocaine) in form of suspension, spray, colloid, foam and/or aerosol. Also the source gas may include one or more anesthetic in disclosed forms.

The source gas and/or secondary gas may be delivered adjacent to energy delivery element generating plasma by gas supply before, during and/or after plasma generation. For example, source gas may be supplied to the energy delivery element using radiofrequency energy for plasma generation even when the energy delivery element is not operating. Flow rate of the source gas may be in the range of 0.005 dm³/min to 500 dm³/min, more preferably in the range of 0.01 dm³/min to 200 dm³/min, most preferably in the range of 0.05 dm³/min to 100 dm³/min.

The device may include at least one plasma source, which may be of any type and may generate any mentioned type of plasma.

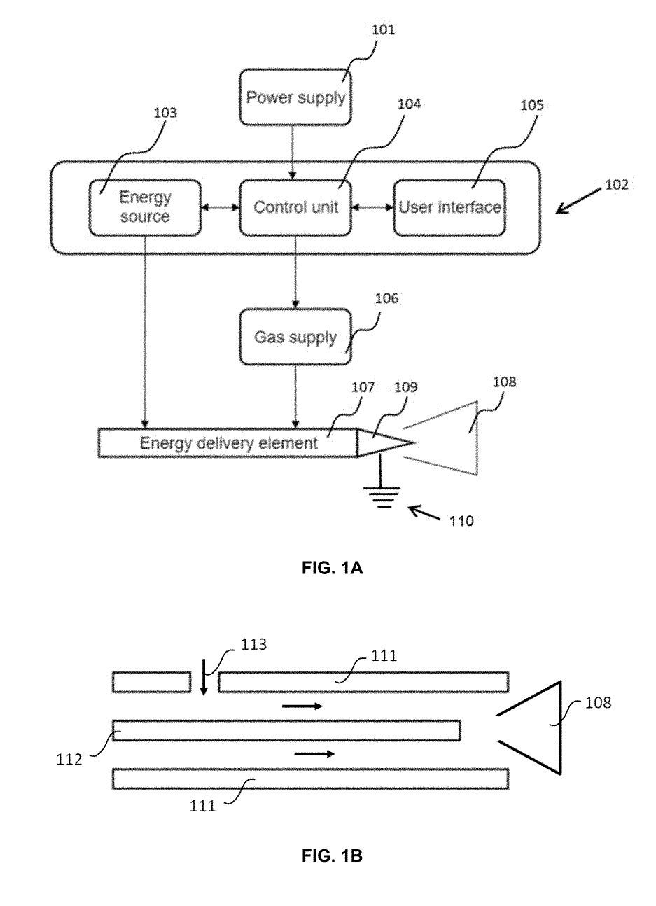

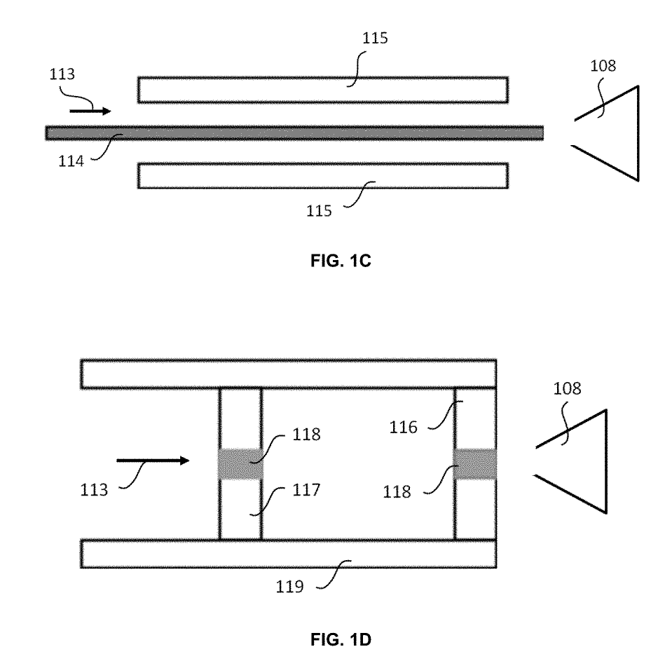

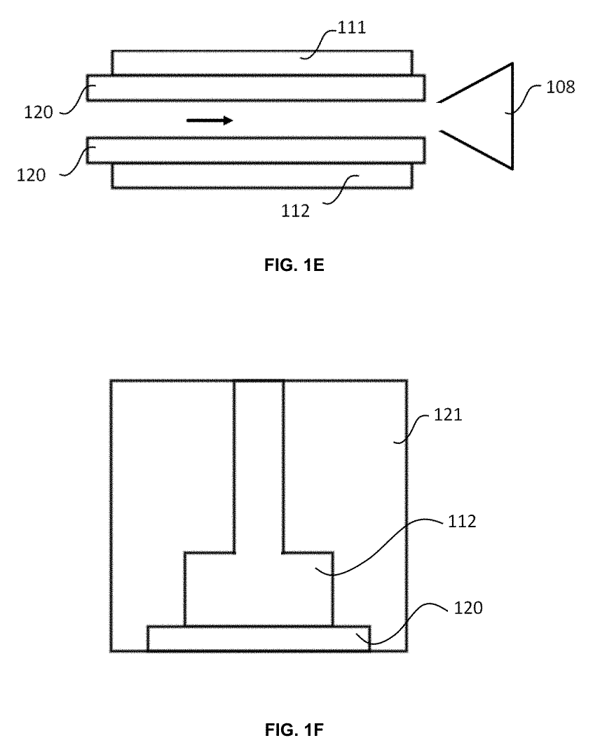

The plasma may be generated by radio frequency. A simplified example of plasma source is illustrated in FIG. 1A showing the plasma source representing plasma jet. The plasma source may have a case 102 which may include an energy source 103, a control unit 104 and a user interface 105. Control unit 103 may be connected to power supply 101. The energy source 103, connected to control unit 104, produces output energy. The power supply may include an electricity network and/or at least one battery. If used, the battery may be part of the case. Output energy is transferred from energy source 103 to energy delivery element 107. The energy delivery element 107 may be coupled to a grounding 110. The exemplary plasma source includes gas supply 106 which delivers gas to the area adjacent and/or around the energy delivery element 107. The gas supply is controlled by control unit 104 which may receive instructions from the user interface 105. The gas supply may be a part of the case 102. The energy delivery element creates an electric field in the region of its ending 109 of the energy delivery element 107 and the gas from the gas supply 106 passes through the electric field, where plasma 108 is generated. The energy delivery element 107 and the ending 109 may be parts of the case 102.

The treatment device may have at least one applicator which may include the plasma source and/or energy source. Furthermore, the applicator may include one or more energy delivery elements providing radiofrequency and/or plasma. Optionally, the plasma source may be located on a separate applicator, while the energy source may be located on another separate applicator, which is used with the first applicator. The device may apply plasma before, during and after application of the energy.

Plasma may be delivered by plasma source represented by plasma jet shown on exemplary FIG. 1B. Plasma jet may include two electrodes between which a gas flows. The outer grounded electrode 111 is grounded while the active electrode 112 creates a discharge generating plasma 108. Arrows 113 represent the flow of the gas. In this configuration, plasma may be generated helium, argon, nitrogen, oxygen, air and/or their mixtures. Mixture of gases may include mixture of argon with up to 5% of oxygen. Alternatively mixture of gases may include mixture of helium with up to 5% of oxygen. Flow rate of the gas may be in the range of 0.1 dm³/min to 50 dm³/min, more preferably in the range of 0.5 dm³/min to 35 dm³/min, most preferably in the range of 1 dm³/min to 25 dm³/min. Frequency of radiofrequency may be in the range of 0.5 MHz to 25 MHz, more preferably in the range of 1 MHz to 20 MHz, even more preferably in the range of 5 MHz to 20 MHz, most preferably in the range of 10 MHz to 15 MHz. Frequency of radiofrequency may be about 13.56 MHz. Voltage between the electrodes may be in the range of 50 V to 350 V, more preferably in the range of 80 V to 300 V, most preferably in the range of 100 to 250 V. In another configuration voltage between the electrodes may be in the range of 0.5 kV to 15 kV, more preferably in the range of 0.8 kV to 10 kV, most preferably in the range 1 kV to 8 kV.

Plasma may be delivered by a plasma source represented by plasma needle shown on the FIG. 1C. The plasma needle includes an active electrode in form of a metal strand 114 inside of a tube (e.g. Perspex tube). An end portion of the metal strand 114 is not covered by the tube 115 and generates plasma 108. In this configuration, plasma may be generated with helium, air, nitrogen, hydrogen and/or their mixture. Mixtures of gases may include mixtures of helium with nitrogen, hydrogen and/or argon. Alternatively mixtures of gases may include mixture of helium with up to 10% of oxygen. Flow rates of the gas may be in the range of 0.05 dm³/min to 10 dm³/min, more preferably in the range of 0.1 dm³/min to 7 dm³/min, most preferably in the range of 0.2 dm³/min to 5 dm³/min. Frequencies of the radiofrequency energy may be in the range of 0.5 MHz to 25 MHz, more preferably in the range of 1 MHz to 20 MHz, even more preferably in the range of 5 MHz to 20 MHz, most preferably in the range of 10 MHz to 15 MHz. The frequency of radiofrequency may be about 13.05 MHz. Voltage between the electrodes may be in the range of 50 V to 350 V, more preferably in the range of 80 V to 300 V, most preferably in the range of 100 to 250 V. In another configuration voltage between the electrodes may be in the range of 0.5 kV to 15 kV, more preferably in the range of 0.8 kV to 10 kV, most preferably in the range 1 kV to 8 kV.

Plasma may be generated by a plasma source represented by plasma pencil shown on the FIG. 1D including of a dielectric tube 119 were the active disc electrode 116 and grounded disc electrode 117 are inserted. Both electrodes have holes 118. Plasma 108 may be generated by application of voltage pulses between two electrodes while a gas may be injected through the holes of the electrodes. In this configuration, plasma may be generated using helium, air, nitrogen, hydrogen and/or their mixture. Flow rates of the gas may be in the range of 0.1 dm³/min to 25 dm³/min, more preferably in the range of 0.5 dm³/min to 18 dm³/min, most preferably in the range of 0.8 dm³/min to 15 dm³/min. The frequency of the radiofrequency waves may be in the range of 0.3 kHz to 50 kHz, more preferably in the range of 0.5 kHz to 25 kHz, most preferably in the range of 0.8 kHz to 20 kHz, Voltage between the electrodes may be in the range of 0.3 kV to 35 kV, more preferably in the range of 0.5 kV to 25 kV, most preferably in the range of 0.8 kV to 20 kV.

Plasma may be generated by plasma source represented by arrangements for dielectric barrier discharge. Exemplary arrangement is shown on FIG. 1E, where the arrangement may include active electrode 112 and grounded electrode 111 covered with dielectric barrier 120, where the plasma 108 may generated by discharge between the electrodes. Frequency of radiofrequency may be in the range of 1 Hz to 800 Hz, more preferably in the range of 5 Hz to 600 Hz, most preferably in the range of 10 Hz to 500 Hz,

Plasma may be generated by a plasma source represented by a floating electrode shown in FIG. 1F. Active electrode 112 may be placed on dielectric barrier 120 (e.g. quartz). Case 121 may be formed from quartz or Teflon (fluorine resins). In this configuration, plasma may be generated from air between the dielectric barrier 120 and the tissue. The frequency of the radiofrequency waves may be in the range of 5 Hz to 5 MHz, more preferably in the range of 50 Hz to 3 MHz, most preferably in the range 100 to 2 MHz. Plasma power may be in the range of 0.01 W/cm² to 2 W/cm², more preferably 0.03 W/cm² to 1 W/cm², most preferably in the range of 0.05 W/cm² to 0.8 W/cm²

Optionally an individual plasma treatment device containing only a plasma source may be provided for e.g. disinfection of tissue surface, tissue shaping, healing process improvement, decreasing amounts of bacteria and microorganisms and/or coagulation. The same treatment procedure may be provided with any of embodiments described above.

The device may include at least one energy source. The energy may be delivered by at least one energy delivery element. Optionally, the energy delivery element may act as the energy source. The radiofrequency waves may be delivered to tissue via at least one electrode or antenna which may be placed in proximity to treated tissue in non-contact, indirect contact and/or direct contact, where direct contact may include invasive application of the energy. At least one energy delivery element may be monopolar and/or unipolar. At least two energy delivery elements may be bipolar, where the reference energy delivery electrode may be on the body of the patient and/or on the applicator. When in a monopolar mode, the temperature gradient may be large while under electrodes in bipolar mode there may be little or no temperature gradient.

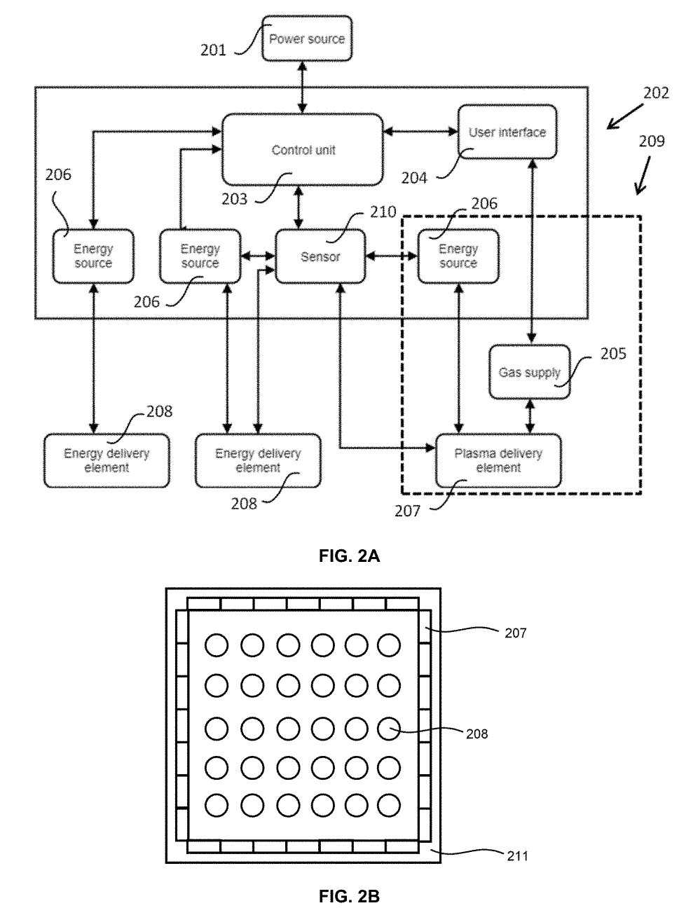

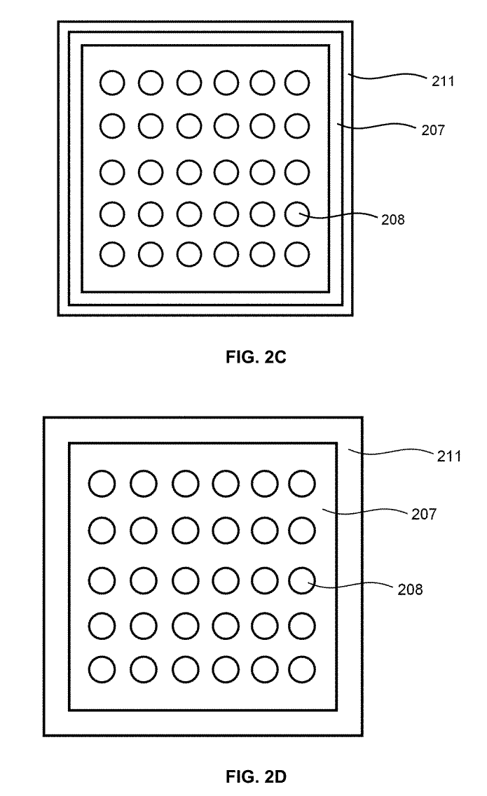

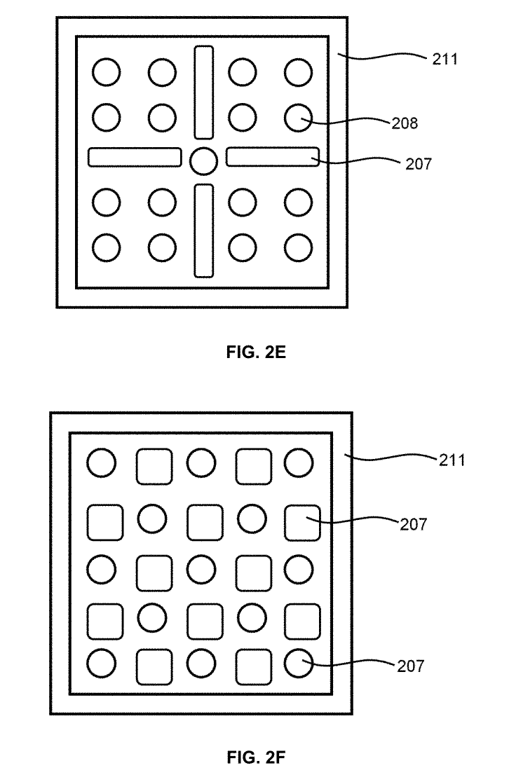

In FIG. 2A power supply 201 may include power grid and/or at least one battery, which may be part of the case 202, that is, the power supply 201 may be part of the case 202. The treatment device may include at least one control unit 203 and user interface 204. Control unit may provide control of at least one of the energy sources 206. A plurality of energy sources 206 may be located on the same applicator. Optionally, in the case of more than one applicator, at least one energy source may be located on one or more of the applicators. The energy source provides e.g. mechanical energy or electromagnetic energy. The gas supply 205 may be placed inside and/or outside case 202. The energy source together with the gas supply form a plasma source 209 shown in dotted line. Energy is transferred from the energy source to at least one energy delivery element 208. According a preferred embodiment the energy delivery element may be an electrode of any shape, e.g. needle, protruding element, element aligned with the applicator surface, element immersed into the applicator or a light guide energy delivery element. However, the mechanical energy may be delivered by vibrational element (e.g. plate, percussion element) and/or piezo element. Magnetic energy may be delivered by magnetic coil. In case of plasma generation (e.g. cold plasma), gas may be delivered from gas supply 205 to the plasma delivery element 207. The plasma delivery element 207 may be the energy delivery element generating plasma, as shown on FIG. 1. In an alternative embodiment the energy delivery element 107 may provide energy generating thermal damage, and later may be used for plasma generation. The energy delivery element may be moved during the operation of the device for this purpose. At least one part of the treatment device is illustrated as an external part (e.g. energy delivery elements) or may be part of case 202. The device may include sensor 210 communicating with control unit 203, one or more energy sources 206, energy delivery element 208 and/or plasma delivery element 207. The applicator may include a tip including energy delivery elements. The tip may be exchangeable. Exchangeable tip may include memory storing information about impedance limit values, amount of remaining pulses and/or type of energy delivery elements in the tip, Gas supply may be replaced by water supply.

The device may include a source of negative and/or positive pressure which may apply pressure on the tissue before, during and/or after the treatment. Source of negative and/or positive pressure may be a vacuum pump. Negative pressure may create skin protrusion which may bring the tissue into the contact and/or near the energy delivery elements and/or eliminate the treatment of the tissue on the unwanted site (e.g. near bones, joints and/or nerves). It may also provide analgesic effect. Positive pressure may provide analgesic effect or decrease blood flow. Negative pressure may be generated by a source of negative pressure e.g. a pump. The negative pressure may be in the range of −100 Pa to −2 MPa, more preferably in the range of −3000 Pa to −400 kPa, most preferably in the range of −4000 Pa to −100 kPa. Deflection of the tissue caused by negative pressure may be in the range of 0.3 mm to 80 mm, more preferably in the range of 0.5 mm to 60 mm, even more preferably in the range of 1 mm to 50 mm, most preferably in the range of 1.5 mm to 35 mm. The applied negative pressure may be continual or pulsed.

Continual pressure means that the pressure amplitude is continually maintained after reaching the desired negative pressure. The pulsed pressure means pressure where the pressure amplitude varies during the therapy. Use of pulsed pressure my decrease inconvenience related to negative pressure by repeating pulses of tissue protrusions at one treated site, when the energy may be applied. The duration of one pressure pulse may be in the range of 0.1 s to 60 s, more preferably in the range of 0.1 s to 30 s, most preferably in the range of 0.1 to 20 s wherein the pulse means duration between the beginnings of successive increases or decreases of negative pressure values. Methods may include treatment of the tissue during continual negative pressure. However, the treatment may also include pulsed pressure.

The method of the treatment may include cooling of the tissue. Tissue may be cooled to a temperature equal to or less than a predefined temperature (e.g. −10° C., −5° C., 0° C., 5° C., 10° C., 15° C., 20° C., 25° C.) before, during and/or after the treatment. The cooling may be provided by a source and/or secondary gas and/or contacting elements such as a thermoelectric cooler. Thermoelectric cooler may be in direct contact with the tissue or it may indirectly cool the tissue through one or more other elements (e.g. a water conduit or metal element).

A spacer may be provided between the tissue surface and the treatment device. This spacer may be used as a mechanical support for the needles and/or for controlling the penetration depth. The treatment device may comprise array of electrodes in a different arrangement attached to a base. The base may be a part of treatment device or can be attached to the housing. Each of the electrodes (e.g. needle or electrode) is electrically connected to an RF source. Control module of the treatment device may control delivery of the radiofrequency energy to at least one needle. This control module may also regulate amount of RF radiofrequency delivered to the treated tissue.

The device may include at least one sensor for detecting the state and/or changes of the treatment. The sensor may also measure values of at least one physical quantity related to the source gas, secondary gas, plasma, energy, energy source and/or plasma source. The sensor may measure concentration of the source gas, flow of the source gas, temperature of the plasma, temperature of energy delivery element, plasma energy delivery element and/or device. It may also measure frequency, output, voltage of the energy source, current of the energy source and/or energy flux. The sensor may also measure at least one physical quantity related to the tissue e.g. temperature of one or more tissue layers, impedance, water content, phase angle of delivered and/or reflected energy, density of the tissue and/or pressure applied by the device on the tissue. The method may include sensing of different physical quantities by one or more sensors and a change of treatment parameters (e.g. position of the needles, temperature of the needles, duration of the energy transfer), energy parameters (e.g. output, frequency, energy flux, phase shift) and/or plasma related parameters (concentration of the source gas, concentration of the secondary gas, flow of the source gas, output of energy delivery element creating plasma) according to data provided by one or more sensors. Alternatively, these parameters may be changed according to the operator's needs. Treatment and operation of the device may also cease by immediate stop of energy transfer and/or retraction of the needles out of the tissue and ceasing plasma generation.

The sensor may be an acoustic, vibration, chemical, electric, magnetic, radio, flow, navigation, positional, optical, imaging, pressure, force, density, temperature, impedance, current, Hall, Doppler and/or proximity sensor. The sensor may also be a gyroscope, capacitive displacement sensor, thermographic camera, ion selective electrode, pH electrode, and the like.

The method may include application of the plasma on the tissue or directly into the tissue. The method may include application of RF energy by needles and/or protruding elements with the application of plasma. Plasma may be applied before, during and/or after the application of the energy. Application of plasma before the application of the energy may provide e.g. disinfection of the tissue from microorganisms and viruses. Application of plasma during the application of energy may provide e.g. improving healing process, reducing pain, coagulation improvement, treatment improvement, prevention of presence of microorganisms and viruses, wound healing initiation and enhancement, cell adhesion improvement and/or cooling of the tissue. Application of plasma after treatment may improve the healing process, prevent inflammation by decreasing amounts of bacteria and microorganisms, influence the biological behavior of epidermal and dermal cells and/or decrease pain.

The method of treatment may include one or more treatment actions. Treatment action may provide treatment to one treatment spot. Treatment spot may be treated by one or more treatment actions. Treatment spot may be defined by the dimensions and/or shape of the surface of the tip facing the tissue. FIGS. 2B-2F shows exemplary embodiments of the surface of the treatment tip facing the tissue including arrangements of the energy delivery elements and plasma delivery elements. FIG. 2B shows plasma delivery elements arranged around the energy delivery element 208. Rim 211 may form the boundary of the surface facing the tissue. FIG. 2C shows one plasma delivery element 207 which is larger than the area where the energy delivery elements are located. FIG. 2D shows a plasma delivery element 207 forming the surface from which the energy delivery element 208 are applied to the tissue. FIG. 2E shows an arrangement of plasma delivery elements 207 with energy delivery elements 208 where the position of plasma delivery elements 207 may provide plasma to most of the surface of the treated tissue. The same effect may be achieved by the arrangement shown in FIG. 2F, where the energy delivery elements 208 are close to plasma delivery elements 207. The treatment spot may be defined as the inner area including all of the energy delivery elements 208 and/or plasma delivery elements 207. The treatment spot may be in the range from 0.1 cm² to 100 cm², more preferably in the range from 0.25 cm² to 25 cm², even more preferably in the range from 0.5 cm² to 10 cm², most preferably in the range of 0.75 cm² to 6 cm². During treatment action, plasma may be applied during different steps of application of energy delivery elements, as described below. Application of the plasma may include supply of the source gas and/or secondary gas adjacent to tissue, generating of the plasma by energy delivery element and/or delivery of the plasma.

A treatment combination using plasma and energy may include a combination of electromagnetic energy with cold plasma. This combination may use radiofrequency waves and a cold plasma source, wherein the radiofrequency source may be used for generation of radiofrequency energy and/or generation of cold plasma. Alternatively, the plasma may be generated by a different radiofrequency source. The frequency of the radiofrequency energy may be changed during the treatment.

Radiofrequency is commonly used for treatment of at least one tissue problem e.g. cellulite, scar, wrinkles and excess of adipose tissue. Invasive applications of radiofrequency may cause open wounds, while non-invasive application of radiofrequency energy may cause overheating of separated volumes inside tissue. Tissue may be also overheated and/or burned by operator's mistake when the applicator is incorrectly positioned near the treated tissue. Furthermore commonly used methods of radiofrequency treatment are used to ablate and/or coagulate treated tissue, which may be painful. Combinations of the radiofrequency with cold plasma may provide further treatment of those problems and provide beneficial improvement of wound healing.

Radiofrequency energy may be delivered to treated tissue (e.g. skin, dermis, hypodermis, adipose tissue) via energy delivery elements e.g. electrodes. Electrodes may deliver energy in monopolar, multipolar, unipolar and/or bipolar modes to create thermal damage in the treated tissue. Frequencies of radiofrequency energy delivered to the tissue may be in the range of 80 kHz to 5 GHz, more preferably in the range of 0.2 MHz to 500 MHz, even more preferably in the range of 0.3 MHz to 50 MHz, most preferably in the range of 0.4 MHz to 30 MHz. Transfer of radiofrequency energy may be provided for a specified time, referred as RF pulse duration, which may be in the range of 0.1 ms to 2500 ms, more preferably in the range of 0.5 ms to 2000 ms, most preferably in the range of 1 ms to 1500 ms. Current density of radiofrequency energy delivered by one energy delivery element may be in the range of 1 Ampere/cm² to 300 Ampere/cm², more preferably in the range of 5 Ampere/cm² to 200 Ampere/cm², most preferably in the range of 10 Ampere/cm² to 150 Ampere/cm². Energy delivery element may have surface contacting tissue in the range of 100 μm² to 50 mm², more preferably in the range of 250 μm² to 25 mm², even more preferably in the range of 500 μm² to 50 mm², most preferably in the range of 5000 μm² to 160000 μm². Radiofrequency energy may be delivered to treated tissue by electrodes represented by protruding elements not penetrating the tissue. In this case the electrodes may have a blunt ending. Energy delivery element having blunt ending may have surface contacting tissue in the range of 500 μm² to 250000 μm², more preferably in the range of 1000 μm² to 200000 μm², even more preferably in the range of 2000 μm² to 180000 μm², most preferably in the range of 5000 μm² to 160000 μm². Also, the blunt ending may have a radius of curvature of at least 0.05 mm. Diameter of the surface contacting tissue of one protruding element may be in the range of 25 μm to 1500 μm, more preferably in the range of 50 μm to 1000 μm, even more preferably in the range of 80 μm to 800 μm, most preferably in the range of 100 μm to 600 μm. Optionally, at least one electrode may be represented as a needle or it may be represented by array and/or matrix of needles. The needles may be attached to and/or penetrate tissue. Penetration depth may be in the range of 10 μm to 10000 μm, more preferably 25 μm to 8000 μm, most preferably 100 μm to 6500 μm. The penetration depth may be set to series of specific value e.g. 1000 μm, 1500 μm, 2000 μm and/or 2500 μm. Interval between specific values may be in the range of 25 μm to 750 μm, more preferably 50 to 600 μm, most preferably 300 μm to 550 μm. Surface of one needle electrode which may be in contact with the tissue may be in the range of 0.05 mm² to 20 mm², more preferably in the range of 0.08 mm² to 15 mm², most preferably in the range of 0.1 mm² to 12 mm². When the needle electrodes are insulated, the insulation layer may have thickness in the range of 1 μm to 150 μm, more preferably in the range of 3 μm to 100 μm, most preferably in the range of 5 μm to 70 μm, most preferably in the range of 8 μm to 50 μm. Diameter of one needle electrode may be in the range of 0.05 mm to 1 mm, more preferably in the range of 0.1 mm to 0.8 mm, most preferably in the range of 0.15 mm to 0.5 mm. At least one needle located in the array and/or matrix may have a different length than other needles. At least one electrode may contain a system for delivery medicaments e.g. analgesics and/or may be provided as a hollow needle to apply a cold plasma directly to the tissue.

Methods of treatment of may include following steps: positioning of the applicator including one or more energy delivery elements adjacent (e.g. in contact) to the tissue; transferring of the energy to the tissue by the energy delivery element, lifting of the applicator including one or more energy delivery elements, positioning of the applicator including one or more plasma source, generating of the plasma using one or more plasma source.

Method of treatment may include following steps: positioning of the applicator including one or more energy delivery elements adjacent (e.g. in contact) with the tissue; transferring the energy (e.g. radiofrequency energy) into the tissue; delivering the source gas and/or secondary gas from gas supply adjacent to another and/or the same energy delivery element and generating of plasma by energy delivery element.

Positioning of the device may include application of the tip on the tissue, where the separating element creating a rim may touch the surface of the tissue. Operator and/or device may apply the tip with such positive pressure, that the rim may press the borders of treatment spot such that the tissue inside the rim may be protruded. Alternatively, negative pressure may be applied and tissue inside the rim may create protrusion. When the energy delivery elements are needle electrodes, positioning may include penetration of the tissue by extending of the needle electrodes. When the energy delivery elements are protruding elements, positioning may include creation of protrusion by protrusion elements.

Transferring the energy (e.g. radiofrequency energy) into the tissue may include application of the energy by the energy delivery elements where the energy may cause thermal damage. Thermal damage may include ablation and/or coagulation of the tissue. Energy may be delivered in a pulsed or continuous manner. When the energy delivery elements are needle electrodes, retraction of the needle electrodes from the tissue may follow the transfer of the energy.

The next step may be delivery of the source gas and/or secondary gas from a gas supply adjacent to the energy delivery element and generation of the plasma. Source gas may be delivered and plasma may be generated by a designated plasma generator (i.e. a plasma delivery element) which is distinct from energy delivery element providing energy causing thermal damage. Also, source gas may flow through the energy delivery element and plasma may therefore be generated by the energy delivery element used for transfer of energy into the tissue and causing thermal damage.

The order of the steps may be changed. Some of the steps may be omitted or be repeated. Source gas may be delivered and plasma may be generated delivered before, during and/or after the energy delivery elements are positioned in or on the tissue. Also plasma may be generated before, during and or after the presence of the needle electrodes in the tissue. Plasma may be generated more than one time during treatment of one treatment spot. Generation of the plasma before and/or after the penetration of the tissue may provide disinfection and/or cooling to the tissue surface and/or energy delivery elements. Generation of the plasma during the presence of the energy delivery element on and/or in the tissue may be provided by plasma delivery element above the tissue and/or by energy delivery elements in contact with the tissue. When the plasma is generated by a plasma delivery element above the tissue, plasma may provide disinfection and/or tissue damaged by energy. When the plasma is generated by energy delivery elements located in the tissue, plasma may provide disinfection, wound healing promotion and/or cooling to the tissue.

Application of negative pressure may include deflection of the tissue by negative pressure and measuring of the contact and/or treatment proximity of the deflected tissue to the energy delivery elements. Treatment proximity may be in the range of 0.1 mm to 5 cm, more preferably in the range of 1 mm to 4 cm, most preferably in the range of 3 mm to 3.5 cm. Contact and/or treatment proximity may be measured by one or more sensor e.g., ultrasound sensor, optical sensor, capacitive sensor, impedance sensor, temperature sensor, Doppler sensor and/or pressure sensor. When the contact and/or treatment proximity is not sufficient, negative pressure may be increased. Also, when the contact and/or treatment proximity is not sufficient, the actuator may move the applicator and/or tip including energy delivery elements close to the deflected tissue. Applicator and/or tip including energy delivery elements may be moved in the range of 0.1 mm to 5 cm, more preferably in the range of 1 mm to 4 cm, most preferably in the range of 3 mm to 3.5 cm. Movement of the applicator and/or tip may be used on body areas including low volume of fat and/or dermis, where the tissue may not be effectively deflected by negative tissue.

The methods of treatment may include measuring temperature of the treated tissue by the temperature sensor. The operator may set the temperature threshold before and/or during the treatment. When the temperature sensor detects the fluctuation of actual measured temperature above or below the set temperature threshold, the device may generate a human perceptible signal, generate plasma, increase or decrease the flow speed of the source gas and/or secondary gas, cease operation and/or change the amount of transferred energy.

The methods of treatment may include measuring the impedance of the tissue by an impedance sensor before, during and/or after the delivery of the energy. The measuring of tissue impedance may include measuring of voltage and/or current of the energy source, where the fluctuation of the voltage and/or current may be used for measurement of tissue impedance. The operator may set the total amount of energy which may be transferred to the tissue during one treatment action and/or during whole treatment. When the energy is being transferred to the tissue (e.g. dermis, hypodermis and/or epidermis), the sensor may measure impedance of the treated tissue layer and the output and/or already delivered amount of energy transferred to the tissue may be derived from such measurement. Alternatively, the sensor may measure impedance of the untreated tissue i.e. tissue layer which is not treated and the output and/or already delivered amount of energy transferred adjacent to the measured tissue layer may be deduced. The response to information measured from the sensor and/or deduced from such information may include limitation of the energy transfer duration in one treatment action and/or not allowing it to proceed with another treatment action during the same treatment. Another advantage of an impedance sensor may include use of subcutaneous anesthetic delivered by injection. Impedance measurement is capable of providing sufficient information about changed impedance of the anesthetized tissue. Method of treatment may include measurement of tissue impedance during initial impedance time interval on the beginning of energy transfer into the tissue. Initial impedance time interval may be in the range of 0.1 ms to 90 ms, more preferably in the range of 0.5 to 80 ms, even more preferably in the range of 0.8 ms to 60 ms, most preferably in the range of 1 ms to 40 ms. When the measured impedance is out of predefined impedance limit values, the energy transfer may be stopped and/or device may provide human perceptible signal. Such control of the energy transfer may prevent pain and/or nonhomogeneous treatment.

The method of treatment may include measuring the output power produced by energy source using a power sensor. The measuring of power may include measuring of voltage and/or current of the energy source. The operator may set the output power of energy which may be transferred to the tissue during one treatment action and/or during whole treatment. Alternatively, the power may be set by device. Power may be measured during initial power measurement interval at the beginning of the energy transfer, during the energy transfer. Initial power measurement time interval may be in the range of 1 ms to 250 ms, more preferably in the range of 3 ms to 200 ms, even more preferably in the range of 5 ms to 150 ms, most preferably in the range of 10 ms to 50 ms. During this interval, the output power of the energy source may be changed to be closer to the power value set by the operator and/or device. The operation of the power source may be controlled by control unit, The change of output power may be executed in steps. After the initial power measurement time limit, the corrected value of the output power may be provided to the tissue.

The method of treatment may include measuring negative and/or positive pressure provided by the device to the tissue during the treatment. The operator may set the pressure threshold before and/or during the treatment. When the pressure sensor detects the fluctuation of actual measured pressure above or below the set pressure threshold, the device may generate a human perceptible signal. Alternatively it may decrease or increase the pressure.

The method of operation may include measuring concentration of the source gas and/or secondary gas by a concentration sensor. The operator may set the concentration threshold of one or more gas used before and/or during the treatment. When the concentration sensor detects the fluctuation of actual measured concentration above or below the set pressure threshold, the device may generate a human perceptible signal, increase or decrease the concentration of measured gas by change of its flow speed, generate plasma and/or cease operation.

The method of treatment may include measurement of penetration depth of the electrodes, e.g. needle electrodes. A sensor capable of such measurement may be an ultrasound sensor and/or camera. The operator may set one or more penetration depth value of needles or different arrays of needles before and/or during the treatment. It should be understood that accuracy of the penetration depth provided by device may not match the set penetration depth value. However, device may move the needles forward to in order to penetrate the tissue into a depth close to a set penetration depth value, where the phrase “close to” refers to a maximum deviation of 20%, more preferably 18%, most preferably 15% from the set penetration depth value. When the concentration sensor detects the fluctuation of actual measured penetration depth above or below the set penetration depth value with disclosed deviations, the device may generate a human perceptible signal, retract the needles, change output, generate plasma and/or cease operation.

Methods of treatment may include use of a tracking system which may provide information usable to distinguish treated and/or untreated treatment spots on the tissue. The tracking system may include one or more contact and/or noncontact sensors, e.g. temperature sensor, ultrasound sensor, impedance sensor, capacitive sensor and/or optical sensor. Ultrasound sensor may provide mechanical wave and measure characteristics of mechanical waves reflected from the tissue. When the sensor is calibrated by measuring the characteristic of mechanical waves reflected by untreated tissue, the treated tissue may be distinguished by one or more changed characteristics of the reflected waves. A capacitive sensor may measure electrical capacity of the tissue and provide information about already treated tissue. An impedance sensor may measure different impedance of the tissue e.g. from the epidermis. An optical sensor may include a sensor measuring the Brewster angle. The treated and/or untreated tissue may be visualized on the user interface in relation to the set treatment pattern, where the device may show the progress in the treatment of the tissue.

The treatment device may comprise equipment for exploring positions of nerves during treatment. It may also detect other structures e.g. sebaceous glands.

According to the first embodiment energy delivery elements may be represented by one or more needle electrode which may be positioned in one or more array. During treatment the needle electrodes may be extended from the device, to deliver energy, application of the plasma and/or other substances and eventual retraction of the needle electrodes back into device at one site of the tissue.

In monopolar needle configurations the heat caused by RF energy may provide treatment around the tip region of the needles. Delivery of the energy may cause at least partial thermal damage in adjacent tissue, which may result in synthesis of new connective tissue in the tissue. The treatment device can be also operated in bipolar mode. In that case, at least one of the needles delivers energy as a positive electrode while at least one other electrode may be a reference electrode. In this configuration the thermal damage may occur around the tips of needles and between two adjacent needles.

Needles spaced in the same array and/or matrices may be positioned within tissue at the different depths. Also an array of needles may have any shape and/or orientation with respect to the tissue. Needle electrodes may therefore be inserted under angle ranging from 1° to 179°, more preferably from 5° to 150°, most preferably from 10° to 100°. Needle electrodes may be inserted perpendicularly into the tissue. In bipolar mode, it may be beneficial to space a first pair of needles closer to each other than from other pairs. Needles may deliver active substance, e.g. analgesics. In some embodiments the device may include targeting apparatus such as light mark.

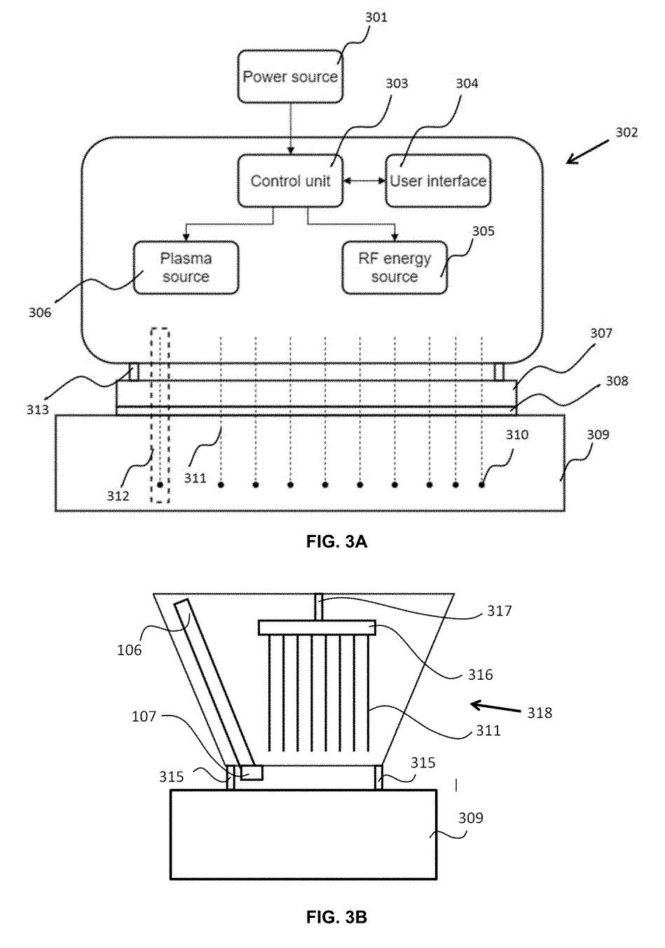

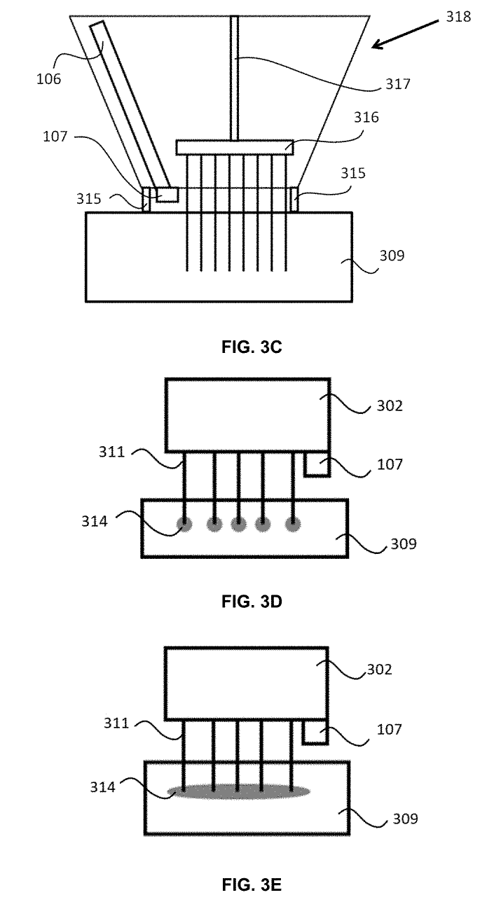

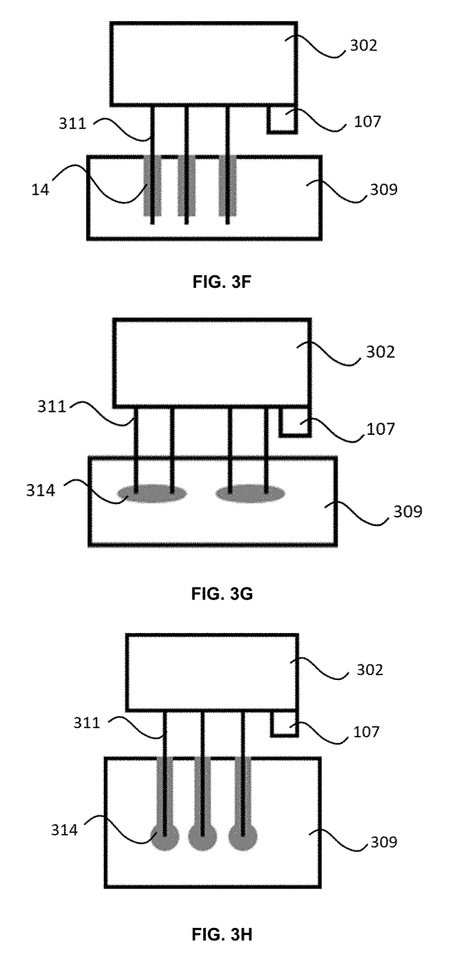

The treatment device shown in FIG. 3A may be provided with plurality of needles 311 attached to the case 302 of the treatment device. The control unit 303 is connected to at least one energy source 305 and at least one plasma source 306, and supplied by power source 301. Each needle 311 may be connected to a control unit 303 which may be also connected to the user interface 304. The control unit 303 may control characteristics of the RF electrical current and control operation of energy source 305 and/or plasma source 306. The device may also contain a spacer 307 which is movably attached to the treatment device via movable mechanism 313. The spacer 307 may contain holes for each electrode (311, 312) to set desired penetration depths.

During the treatment process at least one needle penetrates the tissue 309 until the bottom surface of the spacer 307 contacts the tissue. Energy source 305 may transfer energy via at least one needle to the tissue. Energy may be transferred to the tips 310 of the needles. The spacer 307 may be planar or may be contoured to any shape and/or cooled. Alternatively, the spacer may be omitted.

At least one hollow needle 312 delivering plasma and/or analgesics may be located on the treatment device. Needle 312 may still deliver the radiofrequency energy. All needles or some of the needles may deliver plasma. The treatment device may be also provided with a plate 308 to make the electromagnetic field delivered to the tissue homogenous.

FIGS. 3B and 3C show an exemplary exchangeable tip 318 containing a plurality of needle electrodes 311. These figures are also representative of a device without any exchangeable tips, relative to the areas facing the tissue. FIG. 3B shows the needle electrodes 311 in a retracted position. A plurality of needle electrodes may be coupled to platform 316, which may be a printed circuit board. Platform 316 may be connected to wire 317 connecting the platform 316 with the energy source 305. Alternatively, each needle electrode may be coupled to the energy source by its own wire. The applicator and/or tip may include a gas supply 106 delivering source gas and/or secondary gas to energy delivery element 107. A separating element 315 may form a rim around the applicator's tip facing the tissue. A separating element 315 may be the first and only part contacting the tissue during first contact of the applicator with the tissue 309 and/or after retraction of the needle electrodes from the tissue 309. Contacting the tissue with the rim may be a part of positioning of the applicator adjacent to the tissue.

FIG. 3C shows the needle electrodes 311 in an extended position, when they are inserted into tissue 309 and create microholes along their length. The energy delivery element 107 may generate plasma from source gas supplied by gas supply 106. Source gas and/or plasma may flow between and/or through the needle electrodes 311 and be in contact with the tissue 309. Apart from elements shown, the exchangeable tip may also include one or more energy sources and/or reservoirs of source and/or secondary gas

FIGS. 3D-3H show patterns of thermal damage provided during one treatment step. FIG. 3D shows the needle electrodes 311 inserted to the tissue 309. Delivery of the RF energy by the needle electrodes may create thermal damage 314 localized around the tip of each needle electrode. FIG. 3E shows thermal damage 314 localized between all inserted electrodes. FIG. 3F shows thermal damage 314 localized around the length of each needle electrode 311. Such thermal damage may reach the epidermis. FIG. 3G shows thermal damage 314 localized between pair of adjacent needle electrodes 311. The patterns of thermal damage may be dependent on setting of radiofrequency energy and/or plasma. Furthermore, the patterns may be dependent on presence of an insulating layer covering the needle electrode. For example, a pattern of thermal damage shown in FIG. 3D may be achieved by insulation of needle electrodes apart of their tips. Similarly, pattern of thermal damage shown on the FIG. 3D may be achieved by absence of insulation on the needle electrodes. All the shown types may be combined in order to achieve optimal thermal damage. As shown on FIG. 3H, thermal damage may be located around the tip and length of each needle electrode 314.

The needle electrodes may be extended from the device at high speed. Speed of the needle electrodes extending from the device may be in the range of 0.1 mm/s to 100 mm/s, more preferably in the range of 0.3 mm/s to 50 mm/s, most preferably in the range of 0.5 mm/s to 25 mm/s. Moreover, needle electrodes may move by different and constant speed from the point of entry into the tissue until the needle electrodes reach the predetermined depth. The constant speed of the extension of the needle electrodes from the point of entry until the target depth may be in the range of 0.1 mm/s to 100 mm/s, more preferably in the range of 0.3 mm/s to 90 mm/s, even more preferably in the range of 0.5 mm/s to 75 mm/s, most preferably in the range of 1 mm/s to 25 mm/s. Speed of the needles may be selectable.

The methods of treatment may include sequential application of radiofrequency energy. In one treatment action, more than one RF pulse may be applied to the tissue. In one example, the one RF pulse may applied in one penetration depth of the needle electrodes, then the electrodes may be repositioned into deeper and/or shallower tissue and another RF pulse may be applied by repositioned needle electrodes. In another example, the needle electrodes are positioned in one penetration depth, where one RF pulse may be followed by application of one more other RF pulses. The first RF pulse may have different RF pulse duration than second RF pulse. It is believed that first RF pulse may provide the tissue different electric characteristics (e.g. resistance and/or impedance) leading to lower sensation and the second RF pulse may not be registered by the body in the same level as the first RF pulse.

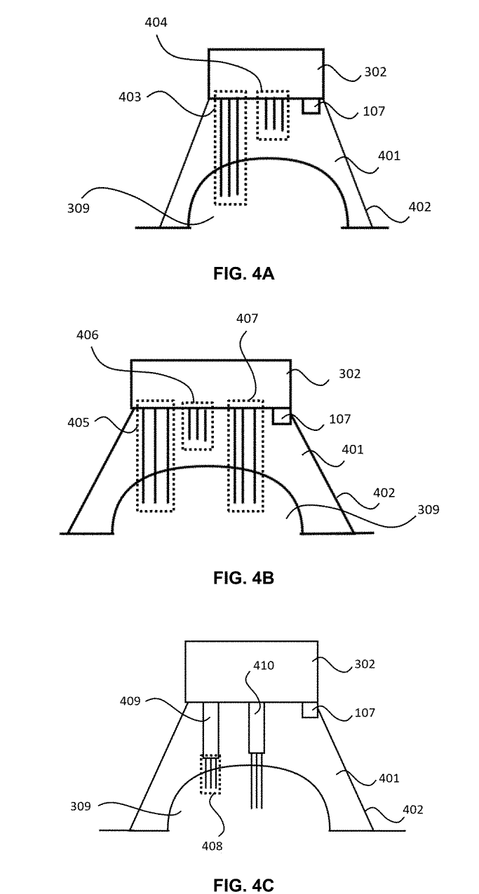

FIG. 4A shows device using negative pressure applied on the tissue. The case 302 may include a source of negative pressure (not shown). The energy delivery element 107 may generate plasma from the gas supplied by gas supply (not shown) in the cavity. First array 403 of needle electrodes is shown penetrating the tissue 309 protruding into cavity 401 by using negative pressure between the outer walls 402. Outer walls 402 may form a rim 211 (shown in FIG. 2B) on contact with the tissue 309. Second array 404 of needle electrodes is shown in a retracted position above the tissue. Any array of needle electrodes may be used as plasma delivery element generating plasma by RF energy. Outer walls 402 may be include needle electrodes extending into tissue. Needle electrodes may penetrate tissue in angle in the range of 1° to 180°, more preferably in the range of 5° to 175°, most preferably in the range of 8° to 160°. FIG. 4B shows similar device with more arrays of needle electrodes. Array 405 is shown penetrating the tissue, array 406 is shown in a retracted position while array 407 is also treating the tissue. Different arrays may treat different spots of the tissue. Retracted arrays 404 and 406 may be retracted inside the device. FIG. 4C shows another embodiment of the device having one or more arrays of needle electrodes (e.g. array 408) positioned on movable platforms 409. Movable platforms 409 may be extended from or retracted into the case 302. Needle electrodes may also be retracted into or extended from the moving platform. During one treatment action the operation, the needle electrodes may penetrate the tissue, provide treatment by RF energy and the plasma may be applied during or after penetration of the tissue. Before the next treatment action, the movable platform may be moved above another treatment spot by motor assembly (not shown) located in the case 302.

Using needle electrodes as energy delivery elements, the method of treatment may include following steps: positioning of the applicator including one or more needle electrodes adjacent (e.g. in contact) with the tissue; Extending the needle electrodes into the tissue; transferring the energy (e.g. radiofrequency energy) into the tissue; retraction of the needle electrodes from the tissue; delivering source gas and/or secondary gas from a gas supply adjacent to another and/or the same energy delivery element and generating plasma by an energy delivery element.

Positioning the device may include application of the tip on the tissue, where the separating element creating the rim may touch the surface of the tissue. The operator and/or a mechanical holding/moving device may apply the tip with such positive pressure that the rim may press the borders of treatment spot such that the tissue inside the rim may be protruded. Alternatively, negative pressure may be applied and tissue inside the rim may create a protrusion.

Extending the needle electrodes into the tissue may include penetrating the tissue surface and positioning of the tips of the needle electrodes into the tissue. Penetration may create microholes in the tissue. Needles may penetrate one or more tissue layers (e.g. stratum corneum, epidermis, dermis, hypodermis, muscle and/or visceral adipose tissue). After extension, tips of the needle electrodes may be located in one tissue layer and/or in the interface of two layers. Alternatively the tips may be located in the interface of two layers and/or between two layers. Extension of the needle electrodes may be executed in more than one separate motions. Needle electrodes may be extended, stopped above the surface of the tissue, and then moved into the tissue.

Transferring energy (e.g. radiofrequency energy) into the tissue may include application of energy by the needle electrodes where the energy may cause thermal damage. Thermal damage may include ablation and/or coagulation of the tissue. Energy may be delivered in a pulsed or a continuous manner.

Retraction of the needle electrodes from the tissue may include vibrations. Needle electrodes may be retracted in one or more separate motions. Needle electrodes may be retracted from the tissue, stopped above the surface tissue (e.g. for plasma generation) and then be retracted into the device.

The next step may be delivery of the source gas and/or secondary gas from a gas supply adjacent to an energy delivery element and generation of plasma. Source gas may be delivered and plasma may be generated by a designated plasma generator (i.e. a plasma delivery element) which is distinct and/or separate from needle electrodes penetrating tissue. Also, source gas may flow through the needle electrodes and plasma may therefore be generated by the needle electrodes used for transfer of energy into tissue and causing thermal damage. Needle electrodes may deliver plasma on the surface of the tissue and/or into the microholes in the penetrated tissue.

The order of the steps may be changed. Some of the steps may be omitted or be repeated. Source gas may be delivered and plasma may be generated delivered before, during and/or after the needle electrodes are positioned in the tissue. Plasma may be generated before, during and or after the presence of the needle electrodes in the tissue. Generation of the plasma before and/or after the penetration of the tissue may provide disinfection and/or cooling to the tissue surface and/or needle electrodes. Generation of the plasma during the presence of the needle electrodes in the tissue may be provided by a plasma delivery element located above the tissue and/or needle electrodes located in the tissue. When the plasma is generated by a plasma delivery element above the tissue, plasma may provide disinfection and/or tissue to tissue damaged by microholes and/or of the ledges of the microholes. When the plasma is generated by needle electrodes located in the tissue, plasma may provide disinfection, wound healing promotion and/or cooling to the tissue.

Needle electrodes combined with plasma may be used for treatment of various tissue problems. More detailed methods of their treatment is disclosed below. For the disclosed methods, the frequency of radiofrequency energy causing thermal damage may be in the range of 0.2 MHz to 10 MHz, more preferably in the range of 0.35 MHz to 8 Mhz, most preferably in the range of 0.5 MHz to 5 MHz. RF pulse duration may be in the range of 25 ms to 1500 ms, more preferably in the range of 50 ms to 1200 ms, most preferably in the range of 100 ms to 1000 ms. Temperature of the treated tissue may be increased in the range of 50° C. to 120° C., more preferably in the range of 55° C. to 110° C., most preferably in the range of 58° C. to 105° C. Current density of radiofrequency energy delivered by one energy delivery element may be in the range of 1 Ampere/cm² to 150 Ampere/cm², more preferably in the range of 5 Ampere/cm² to 125 Ampere/cm², most preferably in the range of 10 Ampere/cm² to 100 Ampere/cm².

Methods of treatment of wrinkles, scars, large skin pores, laxity and/or stretch marks may include positioning of the applicator including one or more needle electrodes in contact with the tissue. Then the needle electrodes may be extended into the dermis and radiofrequency energy may be transferred by the needle electrodes. Radiofrequency energy may cause thermal damage including ablation and coagulation. Thermal damage may lead to damage of connective tissue (e.g., collagen and/or elastin) which may be later replaced by newly synthetized connective tissue. Then the needle electrodes may be retracted from the dermis. Source gas may be delivered by the needle electrodes on the surface of the tissue and generate cold plasma. Also, plasma may be generated and applied to the thermally damaged tissue when the needle electrodes are still in the dermis. Alternatively, source gas may be delivered to the plasma delivery element designated only for the plasma generation, where the plasma may be generated and be applied to the tissue.

Methods of treatment of tattoo removal and eczema treatment may include positioning the applicator including one or more needle electrodes in contact with the tissue. Then the needle electrodes may be extended into the epidermis and radiofrequency energy may be transferred by the needle electrodes. Radiofrequency energy may cause thermal damage including ablation and coagulation. Thermal damage may lead to damage of pigment and/or subsequent immune reaction towards the eczema. Needle electrodes may be retracted from the epidermis. Source gas may be delivered by the needle electrodes on the surface of the tissue and generate cold plasma. Also, plasma may be generated to the thermally damaged tissue when the needle electrodes are still in the epidermis. Alternatively, source gas may be delivered to the plasma delivery element designated only for the plasma generation, where the plasma may be generated and be applied to the tissue.

Methods of treatment of cellulite and/or excess of adipose tissue, may include positioning of the applicator including one or more needle electrodes in contact with the tissue. Then the needle electrodes may be extended into the hypodermis and/or visceral adipose tissue and radiofrequency energy may be transferred by the needle electrodes. Radiofrequency energy may cause thermal damage including ablation and coagulation of adipose tissue. Damaged adipose tissue is removed by body processes after treatment. Needle electrodes may be retracted from the hypodermis and/or visceral adipose tissue. Source gas may be delivered by the needle electrodes on the surface of the tissue and generate cold plasma. Also, plasma may be generated to the thermally damaged tissue when the needle electrodes are still in the tissue. Alternatively, source gas may be delivered to the plasma delivery element designated only for the plasma generation, where the plasma may be generated and be applied to the tissue.

According to another embodiment energy delivery elements may be one or more protruding elements which may be positioned in one or more array. Protruding elements may touch the tissue without any protrusion created by the tissue or they may provide protrusion to the tissue, particularly when the positive pressure is applied by the operator and/or device.

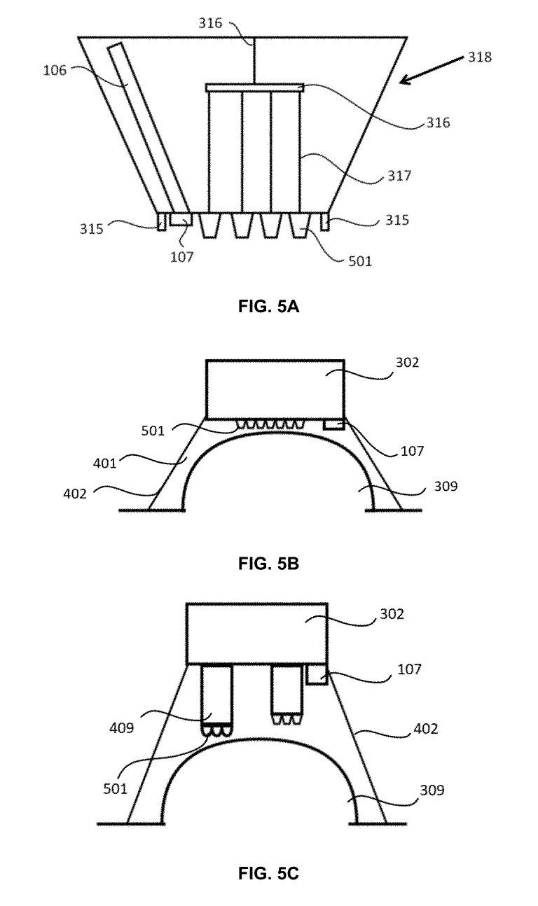

FIG. 5A shows device and/or exchangeable tip using protruding elements 501. Protruding elements 501 may be coupled by wires 317 to platform 316, which may be flex circuit. Platform 316 may be connected to wire 317 connecting the platform 316 with the energy source 305 (shown in FIGS. 3A and 3B). Alternatively, each protruding element 501 may be coupled to energy source by its own wire. The applicator and/or tip may include gas supply 106 delivering source gas and/or secondary gas to energy delivery element 107. A separating element 315 may form a rim around the applicator's tip facing the tissue. Different dimensions and shapes of separating element 315 may define the shape and dimensions of a treatment spot. The separating element creating a rim may be quadrilateral, triangular, pentagonal and/or hexagonal. Separating elements 315 may be shorter than protruding elements. At the beginning of each treatment step, applied positive pressure bring the separating elements 315 close to the tissue, while the protruding element 501 create protrusion in the tissue, Alternatively, the separating element may be longer than protruding elements 501. During application of positive pressure, the separate element 501 may be pressed and protruding elements 501 may contact the tissue. This feature may be used to provide patient with less inconvenience, because rim created by separating elements 315 may limit the amount of applied pressure. Therefore, contacting of the tissue with the rim and application of the negative and/or positive pressure with the help of the rim may be a part of positioning of the applicator adjacent to the tissue. During the treatment, the energy delivery element 107 may generate plasma from source gas supplied by gas supply 106. Source and/or plasma may flow between the protruding elements 311 and be in contact with the tissue 309. Apart from the elements shown, the exchangeable tip may also include one or more energy sources and/or reservoirs of source and/or secondary gas

FIG. 5B shows device using protruding elements 501 together with application of negative pressure. Outer walls 402 may define cavity 401, where the negative pressure may be applied by the source of negative pressure located in case 302. Outer walls 402 may form a rim 211 on the contact with the tissue 309. Negative pressure may draw tissue 309 close to protruding elements 501. Energy delivery element 107 may generate plasma from the source gas delivered by gas supply (not shown). Protruding element 501 may also be used to generate plasma.

FIG. 5C shows another device using protruding elements 501 together with application of negative pressure. Apart from FIG. 5B, the protruding elements 501 in FIG. 5C are shown to be coupled to the moving platform 409. Such configuration allows treatment even when the negative pressure does not draw tissue close enough to the protruding element. It also provides possibility to treat more than one treatment spot during one treatment action.

Using protruding element as energy delivery elements, the methods of treatment may include following steps: positioning of the applicator including one or more protruding elements adjacent (e.g. in contact) with the tissue; transferring the energy (e.g. radiofrequency energy) into the tissue; delivering the source gas and/or secondary gas from gas supply adjacent to another and/or the same energy delivery element and generating of plasma by energy delivery element.

Positioning of the device may include application of the tip on the tissue, where the separating element creating the rim may touch the surface of the tissue. The operator and/or device may apply the tip with such positive pressure, that the tissue inside the rim may be protruded. Alternatively, negative pressure may be applied and tissue inside the rim may create protrusion. Protruding element may touch the tissue and/or create protrusion on the tissue in course of positioning of the device.

Transferring the energy (e.g. radiofrequency energy) into the tissue may include application of the energy by the protruding element where the energy may cause thermal damage. Thermal damage may include ablation and/or coagulation of the tissue. Thermal damage caused by radiofrequency applied by protruding elements may create one or more microholes under each protruding element.

The next step may be delivery of the source gas and/or secondary gas from a gas supply adjacent to energy delivery element and generation of plasma. Source gas may be delivered to and plasma may be generated by a designated plasma generator (i.e. a plasma delivery element) which is distinct and/or separated from the protruding element. Also, source gas may flow through the protruding element and plasma may therefore be generated by the protruding element used for transfer of energy into tissue and causing thermal damage.

The order of steps may be changed. Some of the steps may be omitted or be multiplied. Source gas may be delivered and plasma may be generated and delivered before, during and/or after the protruding elements are positioned in the tissue. Plasma may be generated before, during and or after the presence of the protruding elements in the tissue. Generation of plasma before and/or after the protrusion of the tissue may provide disinfection and/or cooling to the tissue surface and/or needle electrodes. Generation of the plasma during the presence of the protruding element in the tissue protrusions may be provided by plasma delivery element above the tissue and/or protruding elements located in the tissue. When the plasma is generated by plasma delivery element above the tissue, plasma may provide disinfection and/or tissue to tissue damaged by energy. When the plasma is generated by protruding element located in the tissue protrusion, plasma may provide disinfection, wound healing promotion and/or cooling to the tissue.

Protruding element delivering energy combined with plasma may be used for treatment of various tissue problems. More detailed methods of their treatment is disclosed below. For disclosed methods, the frequency of radiofrequency energy causing thermal damage may be in the range of 0.2 MHz to 10 MHz, more preferably in the range of 0.35 MHz to 8 MHz, most preferably in the range of 0.5 MHz to 5 MHz. RF pulse duration may be in the range of 25 ms to 1500 ms, more preferably in the range of 50 ms to 1200 ms, most preferably in the range of 100 ms to 1000 ms. Current density of radiofrequency energy delivered by one protruding element may be in the range of 1 Ampere/cm² to 150 Ampere/cm², more preferably in the range of 5 Ampere/cm² to 125 Ampere/cm², most preferably in the range of 10 Ampere/cm² to 100 Ampere/cm².

Method of treatment of wrinkles, eczema, scars, large skin pores, laxity and/or stretch marks may include positioning of the applicator including one or more protruding element in contact with the tissue. Then the protruding elements may create skin protrusions and transfer radiofrequency energy. Radiofrequency energy may cause thermal damage including ablation and coagulation. Thermal damage may lead to damage of epidermis and/or connective tissue (e.g., collagen and/or elastin) which may be later replaced by newly synthetized connective tissue. Thermal damage may also cause immunological reaction towards the eczema. Then the transfer of the radiofrequency energy may be stopped. Source gas may be delivered by the protruding elements to the surface of the tissue and generate cold plasma. Alternatively, source gas may be delivered to the plasma delivery element designated only for the plasma generation.

According to another embodiment treatment combination of plasma and energy may include a combination of light energy (e.g. laser) with cold plasma. A coherent light energy source may be used for ablative laser skin resurfacing and non-ablative laser skin resurfacing. These methods differ from each other by the depth of thermal damage. Ablative laser skin resurfacing may cause thermal damage to the epidermis and/or dermis. On the other hand, non-ablative laser skin resurfacing may avoid thermal damage in the epidermis. The light may be monochromatic, polychromatic, coherent and/or non-coherent. Energy delivery elements may be one or more light guides ended with one or more transmission elements which may be positioned in one or more array. Light guides may touch the tissue or they may not tissue at all.

Devices may include one or more optical sources. Optical sources may be a laser emitting diode, laser emitting diode, discharge tube, flash lamp, CO2 laser, Q-switched laser, Erbium YAG laser, Nd YAG laser, fiber laser (e.g. Raman-shifted ytterbium-doped fiber laser). An optical source may provide one or more laser beams. One laser beam may be split to plurality of laser beams. The treatment action may include application of more than one laser beams.

The light wavelength may be in the range of 400 nm to 2200 nm, more preferably in the range of 600 nm to 2050 nm, most preferably in the range of 800 nm to 1980 nm. In some embodiments, the light wavelength be in the range of 1025 nm to 1100 nm. In some embodiments, the light wavelength be in the range of 1400 nm to 1420 nm. In some embodiment the light wavelength may be in the range of 1835 to 1940 nm. In some embodiment the light wavelength may be in the range of 1835 to 1880 nm. In some embodiment the light wavelength may be in the range of 1880 to 1940 nm. The wavelength of the applied light may be close to 254 nm, 405 nm, 450 nm, 530 nm, 560 nm, 575 nm, 640 nm, 685 nm, 830 nm and/or 1064 nm. Term “close to” refers to deviation of not more than 20%, more preferably 15%, most preferably 10% from the nominal wavelength.

Pulse energy of the light may be in the range of 0.1 mJ to 100 mJ, more preferably in the range of 0.5 mJ to 75 mJ, most preferably in the range of 1 mJ to 50 mJ. Fluence of the light beam may be in the range of 0.1 J·cm⁻² to 3000 J·cm⁻², more preferably in the range of 1 J·cm⁻² to 1500 J·cm⁻², most preferably in the range of 5 J·cm⁻² to 1000 J·cm⁻². Pulse width may be in the range of 0.01 ms to 1500 ms, more preferably in the range of 0.1 ms to 1000 ms, most preferably in the range of 1 ms to 750 ms. When more than one laser beam is used, the individual beams may be separated by a distance (center to center) of at least 0.1 mm, 0.3 mm, 0.5 mm or 1 mm. Light spot size may be in the range of 0.001 mm² to 600 mm², more preferably in the range of 0.012 mm² to 500 mm², most preferably in the range of 0.01 mm² to 400 mm².

Using light guides as energy delivery elements, the method of treatment may include following steps: positioning of the applicator including one or more light guide adjacent with the tissue; transferring the light to the tissue; delivering the source gas and/or secondary gas from gas supply adjacent to energy delivery element and/or the light guide and generating of plasma.

Positioning of the device may include application of the tip on the tissue, with the separating element creating a rim touching the surface of the tissue. Alternatively, negative pressure may be applied and tissue inside the rim may be pulled into the rim by vacuum. The light guide providing light may touch the tissue and/or be spaced apart from the tissue. The energy delivery element designed only to provide plasma generation may not touch the tissue.

Transferring the energy into the tissue may include application of energy by the light guide where the energy may cause thermal damage. Thermal damage may include ablation and/or coagulation of the tissue.

The next step may be delivery of the source gas and/or secondary gas from a gas supply adjacent to the energy delivery element and generation of the plasma. Source gas may be delivered to and plasma may be generated by a designated to plasma generator (i.e. a plasma delivery element) which is distinct and/or separated from the light guide. Alternatively, the plasma may be generated by the light (e.g. laser).

The order of the steps may be changed. Some of the steps may be omitted or be repeated. Source gas may be delivered and plasma may be generated delivered before, during and/or after the protruding elements are positioned in the tissue. Plasma may be generated before, during and or after the presence of the protruding elements in the tissue. Generation of the plasma before and/or after the light treatment may provide disinfection and/or cooling to the tissue surface and/or transmission element. Generation of the plasma during the light treatment may provide disinfection and/or cooling.

Although the method and device using laser may include treatment by treatment actions, it may also include continuous treatment, when the laser beam is moved over the tissue with continuous delivery of gas and generation of plasma. The laser beam may be provided via a handheld applicator and/or scanning unit.

Treatment by an energy source providing coherent light energy may increase tissue sensitivity and/or cause micro-injuries. Combinations of such energy source with the plasma (e.g. cold plasma) may result in higher comfort of the treated tissue.

Devices may also provide combinations of types of energy together with plasma. The radiofrequency or light may be combined with electric energy. Transfer of electric energy into tissue may provide anesthetic effect. The frequency of the electric energy may be in the range of 0.1 Hz to 200 Hz, more preferably in the range of 1 Hz to 150 Hz, most preferably in the range of 3 Hz to 120 Hz. The frequency of the electric energy may be about 5, 25, 50 and/or 100 Hz. Pulse duration of the electric energy may be in the range 0.1 μs to 100 μs, more preferably in the range of 0.5 μs to 80 μs, most preferably in the range of 1 μs to 60 μs. Electric energy may be applied by energy delivery elements e.g. by contact electrodes.

Using light guides as energy delivery elements, the method of treatment may include following steps: positioning of the applicator including one or more light guide adjacent with the tissue; transferring the light and electric energy to the tissue; delivering the source gas and/or secondary gas from gas supply adjacent to energy delivery element and/or the light guide and generating of plasma.

Positioning the device may include application of the tip on the tissue, where the separating element creating a rim may touch the surface of the tissue. Alternatively, negative pressure may be applied and tissue inside the rim may create protrusion. A light guide providing light may touch the tissue and/or be distant from the tissue. One or more electrodes delivering the electric energy may touch the tissue. The energy delivery element designed only for plasma generation may not touch the tissue.

Transferring energy into the tissue may include application of light by the light guide where the energy may cause thermal damage. Also, electric energy may be applied by electrodes. Thermal damage may include ablation and/or coagulation of the tissue, while electric energy may provide an analgesic effect.

The next step may be delivery of the source gas and/or secondary gas from a gas supply adjacent to the energy delivery element and generation of the plasma. Source gas may be delivered to and plasma may be generated by a designated plasma generator (i.e. a plasma delivery element) which is distinct and/or separated and separate from light guide. Alternatively, the plasma may be generated by the light (e.g. laser).

The order of the steps may be changed. Some of the steps may be omitted or be multiplied. Source gas may be delivered and plasma may be generated delivered before, during and/or after the protruding elements are positioned in the tissue. Plasma may be generated before, during and or after the presence of the protruding elements in the tissue. Generation of the plasma before and/or after the light treatment may provide disinfection and/or cooling to the tissue surface and/or transmission element. Generation of the plasma during the light treatment may provide disinfection and/or cooling.

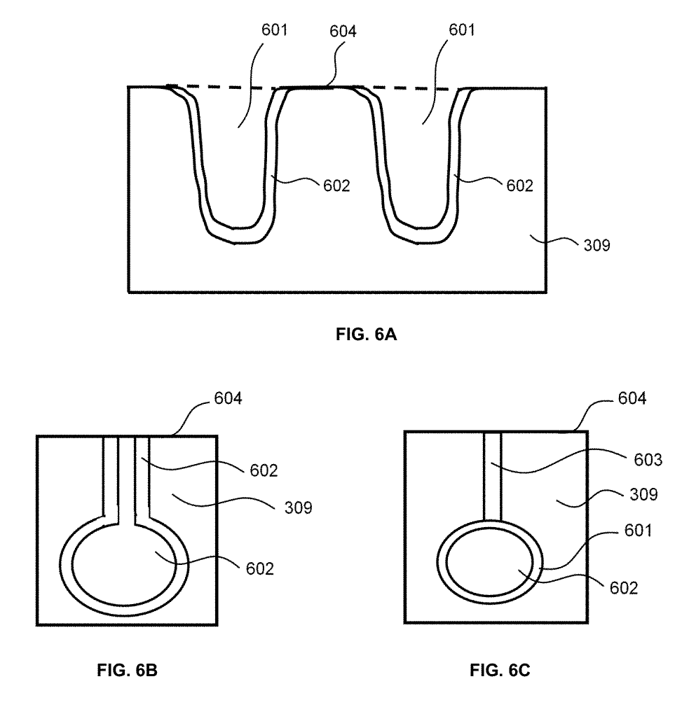

Treatment by energy (e.g. radiofrequency or light) may cause thermal damage which may include ablation and/or coagulation. FIGS. 6A-B show thermal damage which may be caused by application of the energy. FIG. 6A shows tissue 309 with thermal damage which may be caused by protruding elements and/or light. FIG. 6B shows thermal damage which may be caused by a non-insulated needle electrode, while the FIG. 6C shows thermal damage which may be caused by a needle electrode which was non-insulated at the tip, where the thermal damage is located on the end of the microhole 603. Regions of thermal damage may include ablated tissue 601 and coagulated tissue 602. Typically, ablated tissue 601 may be created by direct contact with an energy delivery element and/or close contact with energy, while the coagulated tissue 602 may be created farther from the energy delivery element. Also, coagulated tissue 602 may be created by contact of untreated tissue with ablated tissue 601. The surface of the tissue 604 represents the stratum corneum. Dashed lines in FIG. 6A represent the original line of the surface 601. Volume ratio of coagulated to ablated tissue (mm² to mm²) caused by one energy delivery element may be in in the range of 0.05 to 6, more preferably in the range 0.1 to 4.8, even more preferably in the range of 0.3 to 4, most preferably in the range of 0.5 to 3.5. Thermal damage may be discrete (i.e., limited to the surrounding of the respective energy delivery element). Disclosed surface ratios may provide faster healing because of the presence of coagulated tissue, which may attract a healing response.

In another configuration a plasma source may be alternatively replaced by light source. The light source may induce biostimulation effects of treated tissue resulting in e.g. in faster wound healing. Hence, the device in this embodiment includes energy sources providing RF energy for treatment of tissue and a light source or plasma source providing tissue problem and/or wound healing improvement. Light providing biostimulation effect may be coherent, non-coherent, monochromatic and/or polychromatic.

Light providing biostimulation effect may have a wavelength in the range of about 400 nm to 1200 nm, more preferably in the range from 440 to 1100 nm most preferably in the range from 450 to 1000 nm.

The method of treatment may include a treatment pattern created by treatment actions. The treatment pattern may be represented by movement of the device over the tissue in a predefined manner. The treatment pattern may be divided to plurality of treatment spots, where the device is moved automatically or manually over the spots. The treatment action may be executed in each treatment spot. After the treatment of one spot, the device may be manually and/or automatically moved to the next spot. Alternatively, the device may not be moved and different treated spots may be treated by a different energy delivery element at a different time. During treatment, more than one treatment spot may be treated by one or more energy delivery elements.

The method of treatment may include positioning of the applicator adjacent (e.g. in contact) with the tissue. The positioning may include defining a treatment pattern. During the defining of the treatment pattern, the treatment pattern may be selected from predefined treatment patterns provided by the device. Alternatively, the treatment pattern may be selected from a set of treatment spots created by the device after manual and/or automatic recognition of the tissue problem. In another alternative, the operator and/or patient may create the treatment pattern de novo A treatment spot selected by all disclosed options may be further modified during and/or before treatment. The modification of the treatment pattern may include change of position, amount, shape and/or sequence of treatment spots.

Predefined treatment patterns may be modified before and/or during the treatment. The modification may be done by the operator and/or patient according to their needs. In such case, the modification may be done e.g. using a user interface showing predetermined treatment patterns. The treatment pattern may be represented as one or more segments which may be moved by touching the user interface (e.g. LCD panel) and dragging a segment to a new position. The device may also modify the treatment pattern according to information provided by one or more sensors. For example, when the temperature sensor provides information about increased temperature of the tissue above a temperature threshold, the device may skip one or more next treatment actions, e.g. skip the treatment action provided on the next spot.

The foregoing description of preferred embodiments has been presented for purposes of illustration and description. It is not intended to be exhaustive or to limit the invention to the precise form disclosed. Modification and variations are possible in light of the above teachings or may be acquired from practice of the invention. All mentioned embodiments may be combined. The embodiments described explain the principles of the invention and its practical application to enable one skilled in the art to utilize the invention. Various modifications as are suited to a particular use are contemplated. It is intended that the scope of the invention be defined by the claims appended hereto and their equivalents.

1. A method of tissue treatment including:
positioning an applicator including plurality of energy delivery elements in contact with the tissue with a negative pressure applied to the tissue;
transferring radiofrequency energy into the tissue; and
causing discrete thermal damage to the tissue in areas surrounding the energy delivery elements.

2. The method of claim 1 where the negative pressure brings the tissue into contact with and/or near the energy delivery elements.

3. The method of claim 2 where the negative pressure causes the tissue to deflect by 0.3 mm to 80 mm.

4. The method of claim 3 where the negative pressure is in the range −100 Pa to −2 MPa

5. The method of claim 4 where a current density of the radiofrequency energy delivered by one energy delivery element is in the range of 1 Ampere/cm² to 300 Ampere/cm².

6. The method of claim 5 where the thermal damage includes ablation and coagulation, and a volume ratio of coagulated to ablated tissue (mm² to mm²) caused by one energy delivery element is in the range of 0.05 to 6.

7. The method of claim 6 further including generating a non-thermal plasma having a temperature in the range of −15° C. to 90° C. when it reaches the tissue.

8. The method of claim 7 where the non-thermal plasma is generated in pulse durations in the range of 0.1 nanosecond to 30 seconds.

9. A method of tissue treatment including:
positioning an applicator having a plurality of energy delivery elements in contact with the tissue where a negative pressure is applied to the tissue moving the tissue into contact and/or near the energy delivery elements;
transferring radiofrequency energy into the tissue;
where the radiofrequency energy has a frequency in the range of 0.2 MHz to 500 MHz;
where radiofrequency has current density in the range of 1 Ampere/cm² to 300 Ampere/cm²;
where the negative pressure is in the range −100 Pa to −2 MPa;
where the energy delivery element has a surface contacting tissue in the range of 100 μm² to 50 mm².

10. The method of claim 9 where a deflection of the tissue caused by the negative pressure is in the range of 0.3 mm to 80 mm.

11. The method of claim 9 where the application of the negative pressure is pulsed and where one pressure pulse is in the range of 0.1 to 60 s.

12. The method of claim 9 where an output power of an energy source is changed to be closer to a set power value during a time interval of 3 ms to 200 ms.

13. The method of claim 9 where the radiofrequency energy is provided in pulses and where the duration of one pulse in in the range of 0.1 ms to 2500 ms.

14. The method of claim 9 where the radiofrequency energy causes thermal damage to the tissue including ablation and coagulation, where volume ratio of coagulated to ablated tissue (mm² to mm²) caused by one energy delivery element is in the range of 0.05 to 6.

15. The method of claim 9 further including generating cold plasma having a temperature in the range of 20° C. to 55° C. when it reaches the tissue.

16. The method of claim 16 including generating the cold plasma from a source gas where a flow rate of the source gas is in the range of 0.005 dm³/min to 500 dm³/min.

17. A method of tissue treatment including:
positioning an applicator including a plurality of needle electrodes in contact with the tissue where negative pressure is applied to the tissue;
extending the needle electrodes into the tissue, where the needle electrodes have penetration depth in the range of 10 μm to 10000 μm;
where a speed of extending the needle electrodes extending is in the range of 0.1 mm/s to 100 mm/s;
where a surface of one needle electrode in contact with the tissue is in the range of 0.05 mm² to 20 mm²;
where a diameter of one needle electrode is in the range of 0.1 mm to 0.8 mm;
transferring the radiofrequency energy into the tissue; and
causing thermal damage to the tissue.

18. The method of claim 17 where the needle electrode is insulated by an insulation layer having thickness in the range of 1 μm to 150 μm.

19. The method of claim 17 where the radiofrequency energy delivered by one energy delivery element has a current density in the range of 1 Ampere/cm² to 300 Ampere/cm².

20. The method of claim 17 where the radiofrequency energy has a frequency in the range of 0.2 MHz to 20 MHz.

21. The method of claim 17 where the speed of the needle electrodes is a constant speed from a point of entry until a target depth.

22. The method of claim 17 further including generating a cold plasma having a temperature in the range of 20° C. to 55° C. when it reaches the tissue.

23. The method of claim 22 where the cold plasma is generated by an energy delivery element having a distance from the tissue in the range of 0.1 mm to 15 cm.

24. A method of tissue treatment including:
positioning an applicator including a plurality of protruding elements in contact with the tissue where a negative pressure is applied to the tissue;
where the negative pressure is in the range −100 Pa to −2 MPa
where one protruding element has a surface area contacting tissue in the range of 500 μm² to 250000 μm²;
where a deflection of the tissue caused by negative pressure is in the range of 0.3 mm to 80 mm;
transferring radiofrequency energy into the tissue; and
causing thermal damage to the tissue.

25. The method of claim 24 where the radiofrequency energy has a frequency in the range of 0.2 MHz to 10 MHz.

26. The method of claim 24 where a diameter of a surface of one of the protruding elements contacting the tissue is in the range of 25 μm to 1500 μm.

27. The method of claim 24 where the application of negative pressure is pulsed and where one pressure pulse is in the range of 0.1 to 60 s.

28. The method of claim 24 where the thermal damage is discrete.

29. The method of claim 24 further including generating a cold plasma having a temperature in the range of 20° C. to 55° C. when it reaches the tissue.

30. The method of claim 29 where the cold plasma is generated by an energy delivery element having distance from the tissue in the range of 0.1 mm to 15 cm.
